(19) United States
(12) Patent Application Publication
(10) Pub. No.: US 2025/0302410 A1
(43) Pub. Date: Oct. 2, 2025

(54) MEDICAL IMAGING DEVICE FOR EXAMINATION OF A BODY PART, IN PARTICULAR A MAMMOGRAPHY DEVICE, BIOPSY UNIT AND METHOD FOR ILLUMINATING A BODY PART

(71) Applicant: Siemens Healthineers AG, Forchheim (DE)

(72) Inventors: Sebastian NEWRZELL, Bayreuth (DE); Helmut GOLLWITZER, Erbendorf (DE); Mohd MAHMEEN, Bangalore (IN)

(73) Assignee: Siemens Healthineers AG, Forchheim (DE)

(21) Appl. No.: 19/095,339

(22) Filed: Mar. 31, 2025

(30) Foreign Application Priority Data
Apr. 2, 2024 (DE) 10 2024 203 028.7

(51) Int. Cl. A61B 6/50; A61B 10/02; A61B 90/00

(52) U.S. Cl. CPC A61B 6/502; A61B 10/0233; A61B 90/39; A61B 2090/3908; A61B 2090/3937

(57) ABSTRACT

One or more example embodiments relates to a medical imaging device for examination of a body part of a patient, in particular a mammography device for examination of the female breast, comprising an imaging unit configured to perform an X-ray-based imaging concerning the body part positioned in a positioning area of the medical imaging device; and a biopsy device comprising a biopsy needle configured to obtain a tissue sample from the body part positioned in the positioning area and a light generating device configured to generate light to reach the positioning area.

### CROSS-REFERENCE TO RELATED APPLICATION(S)

The present application claims priority under 35 U.S.C. § 119 to German Patent Application No. 10 2024 203 028.7, filed Apr. 2, 2024, the entire contents of which is incorporated herein by reference.

### FIELD

One or more example embodiments relates to a medical imaging device for examination of a body part of a patient, such as a mammography device for examination of the female breast, comprising an imaging unit, via which an X-ray-based imaging may be performed concerning a body part positioned in the positioning area of the medical imaging device, and a biopsy device comprising a biopsy needle for obtaining a tissue sample from the body part positioned in the positioning area.

### RELATED ART

Medical imaging devices allow the generation of image data, in particular X-ray images of the body part positioned in the positioning area. Medical imaging devices are, for example, used in the context of performing X-ray mammography for early detection of breast cancer. Recently, the method of 3D mammography or tomosynthesis has been increasingly used via an imaging device designed as a mammography device. Here the body part or the breast is X-rayed from different angles, such that multiple X-ray images are generated from different perspectives. This enables both easier detection and improved localization of tissue changes.

If in the context of a preliminary examination or the X-ray imaging a potentially malignant tissue change has been detected and localized, a histological investigation of the tissue concerned is often indicated. To this end, typically via a biopsy needle of a biopsy device which is an in particular detachable component of the medical imaging device, a tissue sample is taken from the affected area. Often to this end, immediately after imaging and while the body part or the breast is still in the positioning area, the biopsy needle is introduced or inserted into the body part and brought to the affected tissue. A tissue sample is then taken via the biopsy needle. To perform the biopsy, local anesthesia is typically needed which must be administered with the greatest possible positional accuracy at the puncture site of the biopsy needle and the area of tissue from where the tissue sample is to be taken. Furthermore, in the context of the preparations for performing the biopsy, it is often necessary that the puncture site is disinfected and, if necessary, for marking purposes, a small cut made, to enable easier and safer insertion of the biopsy needle. The preparations necessary in the context of performing the biopsy, in particular the performance in the context of the local anesthesia of the injection and/or disinfection and/or cutting at the intended puncture site, are often arduous due to the positional accuracy required.

### SUMMARY

One or more example embodiments specifies an improved concept in relation to a medical imaging device, such as with regard to the preparations necessary in the context of an examination or the performance of the biopsy. These preparations concern, for instance, the problems addressed above of the positionally accurate administration of local anesthesia, the necessary prior disinfection and/or the marking of the puncture site via a small cut in the skin.

### BRIEF DESCRIPTION OF THE DRAWINGS

Advantages and details may be seen from the exemplary embodiments described below and from the figures. These show schematically as follows:

FIG. 1 a highly schematic representation of a front view of a medical imaging device according an exemplary embodiment, comprising a biopsy unit according to an exemplary embodiment, a method according to an exemplary embodiment being explained based on this medical imaging device;

FIG. 2 a perspective detailed view of the biopsy unit of the medical imaging device of FIG. 1;

FIG. 3 a perspective detailed view of a retaining element of the medical imaging device of FIG. 1 from above;

FIG. 4 a perspective detailed view of a retaining element of the medical imaging device of FIG. 1 from below;

FIG. 5 a perspective view of a laser source of a marking device of a light generating device of the medical imaging device of FIG. 1;

FIG. 6 a view of two light beams generated via the marking device, and

FIG. 7 a view of a lighting device of a light generating device of the medical imaging device of FIG. 1.

### DETAILED DESCRIPTION

According to one or more example embodiments, this is achieved with a medical imaging device having a light generating device, via which light reaching the positioning area may be generated.

Multiple synergic advantages are achieved in this way. One of these advantages is that light generated via the light generating device is introduced in a targeted manner in the positioning area to illuminate the body part. Since the light generating device is a component of the medical imaging device, a significantly better lighting situation is achieved than is the case in the context of the ambient lighting typically provided. Thus, the lighting provided by the ambient lighting of the body part positioned in the positioning area is comparatively unfavorable since corresponding light sources, for example mounted on the ceiling of a room, typically are too far away from the positioning area for this to create an adequate lighting situation. The problem of shadowing also arises frequently here, caused for instance by the medical staff present and/or components of the medical imaging device. This disadvantage is overcome with the medical imaging device according to one or more example embodiments, due to the light generating device provided specifically for lighting the positioning area. The light generating device is preferably positioned or can be positioned in relation to the positioning area in such a way that light generated and emitted by the light generating device reaches the positioning area in a manner in which it is free of reflection.

A further advantage of the medical imaging device according to one or more example embodiments relates to the fact that this has the imaging unit, the biopsy device and the light generating device all together or at the same time. These devices are provided accordingly at different positions. Therefore it is unnecessary that, for instance following administration of the local anesthesia, the light generating device has to be dismantled so that in its place the biopsy device may be mounted. Not only does this simplify handling of the medical imaging device for the medical staff, but the time taken to perform the procedure is shortened meaning that this is less stressful for the patient.

As already indicated, the medical imaging device according to one or more example embodiments may be a mammography device for examination of the female breast. It is also conceivable that the medical imaging device is a magnetic resonance device, via which for example image data and biopsy samples concerning the liver of the patient may be obtained, or a computed tomography device for examination of the female breast.

According to one or more example embodiments it may be provided that the light generating device comprises a marking device, via which a marker light for targeted marking of a site on the body part positioned in the positioning area may be generated. The marker light impinges on a site on the body part that is important in the context of the further procedure. In the context of this embodiment the improved lighting situation therefore relates not just to the mere illumination of the body part, but also to the specific achievement of the possibility of better orientation in the context of the preparation for the biopsy. Thus, the marking may if necessary designate a site which, in the context of the preparation for the biopsy, i.e. for instance of the local anesthesia to be administered, is relevant and is for instance an ideal puncture site for an injection required to this end. The marker light is preferably concentrated or focused so that it generates the smallest possible light spot or the narrowest possible light or marking line on the body part. The marker light is preferably a monochromatic light, i.e. a laser light.

In relation to the biopsy needle it is preferably provided that this is movable in its longitudinal direction to bring it into a state of insertion in a body part positioned in the positioning area. In particular, the biopsy needle has a straight shape and is repositionable or displaceable or movable in the corresponding longitudinal direction. For this purpose the medical imaging device may have an electromechanical actuator, which is motion-coupled with the biopsy needle. The electromechanical actuator, which is for instance an electric motor, is preferably a linear actuator. Typically, however, it is provided that the biopsy needle is movable in its longitudinal direction not, or not only, by motor, but manually by the doctor. To achieve a corresponding linear-guided movement, the biopsy needle may be movably mounted via a linear guide. The biopsy device preferably comprises a housing. In this respect, with regard to the movement or displacement in its longitudinal direction the biopsy needle may be extendable from the housing or movable with the housing. Particularly preferably the housing is movably mounted via the linear guide. The site marked by the marker light may be the site at which the longitudinal direction of the biopsy needle, i.e. an imagined extension of the biopsy needle, impinges on the body part positioned in the positioning area. Consequently the marker light marks the site on the body part, which the biopsy needle pierces when moving in its longitudinal direction. The diameter of the biopsy needle may be between 3 mm and 5 mm but, for instance depending on the intended purpose in each case, may also deviate from this.

It is conceivable that the marker light is formed by a first light beam, propagating in a fan-like manner within a first propagation plane, and by a second light beam, propagating in a fan-like manner within a second propagation plane, the propagation planes being at an angle to one another, the longitudinal direction of the biopsy needle lying within each of the propagation planes of the light beams. Since the longitudinal direction of the biopsy needle lies within the two propagation planes, which are at an angle to one another, it necessarily follows that the propagation planes intersect in the longitudinal direction of the biopsy needle. Each of the light beams may be generated by a separate laser source. The respective light beam propagates laterally from the marking device. This means that the respective light beam propagates or spreads in a direction of propagation, which for example may be understood to be the central axis, in particular the axis of symmetry, of the fanning light beam. Furthermore, the lateral expansion occurs in a spatial direction perpendicular to the direction of propagation. In relation to the remaining, third, spatial direction, which lies perpendicular to the direction of propagation and to the expansion direction just described, the light beam remains roughly focused or punctiform. The geometric figure describing the propagation of the respective light beam is therefore a two-dimensional cone or an equilateral triangle with the marking device at the apex.

Each of the two propagation planes runs spatially in such a way that the longitudinal direction of the biopsy needle is contained in such plane. Since the two propagation planes are at an angle, in particular perpendicular, to one another, a cross of light results on the body part, the point of intersection of which indicates or marks the puncture site of the biopsy needle, and which is formed by two marking lines, each generated by one of the two light beams. The biopsy needle and the marking device are preferably positioned relative to one another with positional accuracy, i.e. they are immobile in relation to each other, so that the correct marking of the puncture site of the biopsy needle takes place automatically and, apart from any initial setting, without the need for adjustment.

Optionally, the light generating device may have at least one mirror, the direction of propagation of the marker light being deflectable via the at least one mirror towards the positioning area. This enables potentially greater flexibility in relation to the installation position of the marking device. In particular if the marker light is a laser light, the marking device producing or comprising a laser source frequently has an elongated design, in particular cylindrical, the direction of propagation of the marker light running in the longitudinal direction of this laser source. To produce a structure of the light generating device that is as horizontal and flat as possible, in particular a plate-like body of a retaining element explained in more detail further on, the longitudinally designed marking device may be installed correspondingly horizontally. Thus, the direction of propagation before the at least one mirror may run substantially horizontally and after the at least one mirror substantially vertically. A mirror surface of the mirror is tilted here in relation to the vertical direction, for example by 45°. If the biopsy device is repositionable, in particular pivotable, then the explanations or spatial directions just set out relate to a position at which the longitudinal direction of the biopsy needle is vertical, i.e., is pointing downwards in relation to the direction of gravity. The mirror preferably comprises two mirror surfaces, for example arranged in a V shape in relation to one another, one of the two light beams being reflected on each of the mirror surfaces. It is also conceivable for the mirror to have two separate mirror surfaces spaced apart from one another. These mirror surfaces may, like the V-shape, be arranged correspondingly angled in relation to the vertical direction and in relation to the respective other mirror surface but without touching this.

Particularly preferably, it is provided that the light generating device, in particular in addition to the marking device, comprises a lighting device, via which an illuminating light for illuminating the body part positioned in the positioning area may be generated. Via the lighting device an overall improved lighting situation in relation to the body part is achieved. Thus, the lighting device is or comprises preferably at least one light-emitting diode. The lighting device may be an elongated light strip, in the longitudinal direction of which multiple light sources, in particular light-emitting diodes, are arranged. The illuminating light may be polychromatic, in particular white. The geometric figure describing the expansion of the illuminating light may be a three-dimensional cone with the lighting device at the apex. The illuminating light may therefore, in particular compared to the marker light, be more fanned-out or unfocused.

The light generating device may have a retaining element with at least one marking device receiving section for receiving the marking device and/or at least one lighting device receiving section for receiving the lighting device. The retaining element may be a one-piece component, consisting for instance of a plastic. A multi-piece structure is also conceivable, however. The marking device receiving section and the lighting device receiving section are preferably arranged directly adjacent to one another. The retaining element may have a body, on or in which at least one receiving section is arranged or integrally formed. The body may be plate-like and preferably extend along the horizontal plane.

It is conceivable that the marking device receiving section and/or the lighting device receiving section is a receiving compartment recessed into a body of the retaining element or penetrating the body. The geometric form of the receiving compartment is preferably adapted to the geometric form of the marking device or the lighting device. The geometric form of the receiving compartment is in particular cuboid or cylindrical. If both fan-like light beams may be generated via the marking device, it is conceivable that the marking device comprises two laser sources each generating one of the light beams. Each of the laser sources may be arranged in a separate marking device receiving section. The laser sources may be arranged in a common marking device receiving section, in which the mirror for deflecting the marker light may be arranged centrally in an area between the two laser sources facing each other.

Particularly preferably the medical imaging device according to one or more example embodiments comprises a guide element incorporated in a guide element-receiving section of the retaining element, the biopsy needle being positionally stabilized via the guide element. Preferably the guide element engages around the biopsy needle laterally, in particular fully, so that it counteracts a movement or deflection of the biopsy needle transversally in relation to the longitudinal direction. The guide element may be a guide bushing, the longitudinal direction of which corresponds to the longitudinal direction of the biopsy needle. The biopsy needle may, while moving in its longitudinal direction towards the body part, pass through a corresponding through opening of the guide element.

The medical imaging device according to one or more example embodiments may have a retaining device, on which the biopsy device and the light generating device are mounted. The retaining device creates a common carrier for these two devices, and is in particular arranged on or attached, in particular detachably, to a stand or lifting column, and is vertically movable. The biopsy device and/or the light generating device is preferably detachably mounted on the retaining device. Thus, the retaining device has retaining sections for the biopsy device and the light generating device respectively, wherein the respective device can be positioned on the respective retaining section and, for instance while forming a screw and/or clip and/or latching connection, can be attached in a detachable manner to the retaining device.

To achieve a joint repositioning capability of the biopsy device and the light generating device in relation to the positioning area the retaining device is preferably repositionable. In relation to the repositioning capability the height adjustability already mentioned, enabled via the stand, is conceivable. It is also conceivable that the retaining device is pivotable, in particular about a horizontal axis. Thus, for example, the longitudinal direction of the biopsy needle may be pivoted from a vertical into a horizontal position and vice versa. The repositioning of the retaining device takes place in such a way that the positions of the biopsy device and the light generating device relative to one another remain the same. The same therefore applies to the longitudinal direction of the biopsy needle and to the line of intersection of the propagation planes.

The medical imaging device according to one or more example embodiments may have a positioning device arranged at or in the positioning area, with the body part being able to be positioned via the positioning device in the positioning area for performing the examination. Via the positioning device, the body part may preferably be secured in place in the positioning area, so that in this case the positioning device may also be referred to as a securing device. This means that the current position or location of the body part in the positioning area is kept substantially stable or constant, even if the patient may move. The positioning device may have clamping parts, wherein the body part is able to be clamped via the clamping parts, in particular between the clamping parts, for securing in the positioning area. Thus, a first clamping part may be an, in particular horizontal, clamping table and a second clamping part an, in particular horizontal, clamping plate, wherein the body part, for instance the patient's breast, is able to be secured in a clamping manner between the clamping table and the clamping plate. Thus, the medical imaging device may have a vertically displaceable guide carriage attached to the stand which carries the retaining device and the positioning device. In this respect it is conceivable that in relation to the guide carriage, the clamping table is positionally fixed, and the clamping plate is positionally adjustable, in particular height adjustable.

The imaging unit preferably has an X-ray source for generating incident X-ray radiation in the positioning area and an X-ray detector for detecting the X-ray radiation passing through the positioning area. Detector data concerning the X-ray radiation collected via the X-ray detector may be used in the context of the intended imaging. For this purpose, the detector data is transmitted to an evaluation device, which may be connected to the medical imaging device or is a component of the medical imaging device. On the evaluation device software may be implemented, via which the X-ray images may be generated based on the detector data acquired by the X-ray detector. The X-ray detector may be a component of the clamping table.

The X-ray source may be repositionable in relation to the positioning area, so that the imaging may be performed based on various perspectives of the body part. If necessary the X-ray detector is also repositionable. In relation to the repositioning capability a pivoting capability is conceivable. Thus, the X-ray source, and if necessary the X-ray detector, may be pivotable, in particular, about a horizontal pivot axis and/or a pivot axis running through the positioning area.

The following summarizes the procedure in the context of the present invention. Thus, the typical initial situation is that in the context of a medical checkup, in particular for the prevention or early detection of breast cancer, an area in the body part or breast affected by a potentially malignant tissue change is detected meaning that clarification is needed in this regard. In the context of this clarification the medical imaging device according to one or more example embodiments is then used.

To this end, initially, once the body part has been secured in the positioning area, a localization of the affected tissue area is performed via the imaging unit. Based on the correspondingly generated image data or images this area or site may be specified, typically by the doctor. This is performed, for instance, by marking the respective position on the images, for instance by positioning a mouse pointer over this area and then clicking. The software implemented by the evaluation device may then determine and output the associated coordinates, for instance the corresponding x-, y- and z-values.

Then the biopsy needle, is positioned, for instance by pivoting the retaining device, in such a way that its longitudinal axis impinges on this site or coordinates and the subsequent longitudinal displacement of the biopsy needle, during which the biopsy needle enters the body part and is advanced to the site, may take place, without components, for instance the positioning device, standing in the way. The light generation then takes place via the light generating device together with the associated preparations, namely the necessary prior disinfection, marking of the puncture site and positionally accurate administration of the local anesthetic.

Upon completion of these preparations the displacement of the biopsy needle in its longitudinal direction takes place, with this piercing the body part and being moved to the affected site. This displacement of the biopsy needle in the context of the actual performance of the biopsy is typically carried out manually by the doctor. To ensure safe movement control the biopsy needle is displaceable exclusively in its longitudinal direction and therefore has just a single degree of freedom of movement. This limited manual movability of the biopsy needle may for instance be achieved by using a cobot. This term is short for collaborative robot, the corresponding concept in the context of treatment or examination combining manual execution by the medical staff or the doctor and robot-assisted processes.

As soon as, or just before, the biopsy needle is positioned at the intended site, the tissue sample is then taken, for instance in a hollow interior of the biopsy needle. Possible methods in this respect, for instance vacuum or punch biopsy, have been known to the person skilled in the art for many years and are therefore not explained in more detail here.

One or more example embodiments also relates to a biopsy unit for a medical imaging device for examination of a body part of a patient, the medical imaging device comprising an imaging unit, via which an in particular X-ray-based imaging may be performed concerning the body part positioned in the positioning area of the medical imaging device, the biopsy unit having a biopsy device comprising a biopsy needle for obtaining a tissue sample from the body part positioned in the positioning area. The object of example embodiments is achieved with such a biopsy unit, in that this has a light generating device, via which, with respect to a state in which the biopsy unit is mounted on the remaining portion of the medical imaging device, light reaching the positioning area may be generated. All advantages, features and aspects explained in connection with the medical imaging device according to example embodiments are equally transferable to the biopsy unit according to example embodiments and vice versa.

The remaining portion of the medical imaging device means those components of the medical imaging device that are not part of the biopsy unit. The mounting of the biopsy unit on the remaining position of the medical imaging device, for instance on the guide carriage, may be performed via a screw and/or clip and/or latching connection.

In particular in relation to the biopsy unit according to one or more example embodiments it is conceivable that this comprises a retaining device, on which the biopsy device and the light generating device are mounted.

One or more example embodiments also relates to a method for illuminating a body part of a patient. In relation to this method the object is achieved in that a medical imaging device in accordance with the above description is used, the body part being positioned in the positioning area, and light reaching the positioning area being generated via the light generating device. All advantages, features and aspects explained in connection with the medical imaging device according to example embodiments and the biopsy unit according to example embodiments are equally transferable to the method according to example embodiments and vice versa.

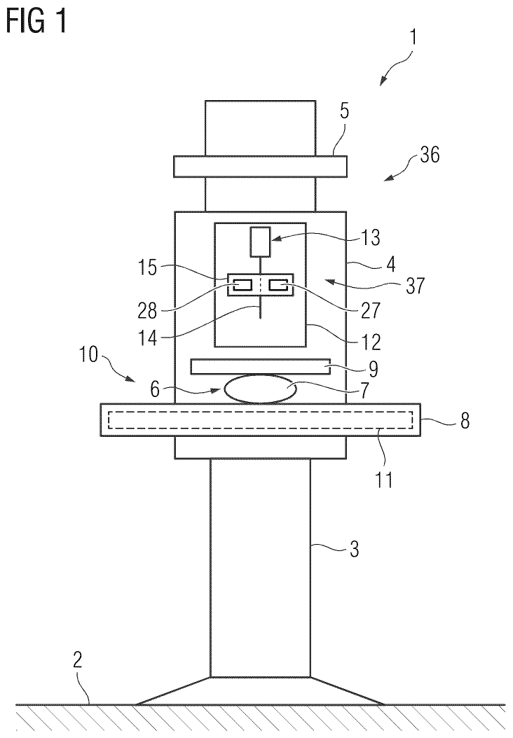

FIG. 1 shows a highly schematic front view of a medical imaging device 1 according to an exemplary embodiment, based on which a method according to an exemplary embodiment is explained. In the situation shown the medical imaging device 1 is positioned on a horizontal surface 2 which is thus perpendicular to the direction of gravity. The medical imaging device 1 shown is a mammography device intended for examination of the female breast, and may be used in the context of X-ray imaging and obtaining a tissue sample.

The medical imaging device 1 comprises a stand 3 designed as a lifting column, on which a guide carriage 4 is arranged. The guide carriage 4 is displaceable and arrestable in a longitudinal direction of the stand 3, i.e. is height-adjustable. Furthermore the stand 3 carries an X-ray source 5 of an imaging unit 36, via which X-ray radiation may be generated. The X-ray radiation generated via the X-ray source 5 occurs in a positioning area 6 in which a body part 7, namely the patient's breast, is positioned. The positioning area 6 is arranged between a clamping table 8 and a clamping plate 9, which form a positioning device 10 attached to the guide carriage 4 and intended for positioning the body part 7 in the positioning area 6. In relation to the guide carriage 4 the clamping table 8 is positionally fixed and in relation to the vertical direction the clamping plate 9 is positionally adjustable in a longitudinal direction of the stand 3. Thus, in the context of the present exemplary embodiment of the method the guide carriage 4 is brought into a vertical position so that the patient may place or position the breast or the body part 7 on the clamping table 8, and then in order to secure the body part 7 the clamping plate 9 is moved downwards so that this is firmly secured via a corresponding clamping effect. The positioning device 10 may therefore also be referred to as a securing device.

In relation to performing the X-ray imaging, the clamping table 8 comprises an X-ray detector 11 of the imaging unit 36, via which the X-ray radiation generated by the X-ray source 5 is detectable after passing through the positioning area 6 and the body part 7, the corresponding detector data being used in the context of the imaging. Via the medical imaging device 1, however, not only is the taking of an individual X-ray image possible, but also the performance of a 3D mammogram, also known as tomosynthesis. To this end the X-ray source 5 is pivoted namely about a horizontal axis perpendicular to the drawing plane with regard to FIG. 1, running through the positioning area 6. This enables imaging to be performed based on different perspectives of the body part 7, to this end the X-ray source 5 being pivoted through angles deviating from the vertical by up to 25° for example. This action allows a depth detection or stereotactic acquisition with regard to the localization of any tissue changes or tumors that may be present in the body part 7.

The guide carriage 4 also carries a retaining device 12, on which in turn a biopsy device 13 with a biopsy needle 14 and a light generating device 15 are arranged. Via the biopsy device 13, often also referred to as a biopsy gun, or the biopsy needle 14, the obtaining of a tissue sample from the body part 7 positioned in the positioning area 6 is enabled. The biopsy needle 14 is for example a hollow needle with a diameter of up to several millimeters, in particular with a diameter of between 3 mm and 5 mm. Via the light generating device 15 a light may be generated which, following emission from the light generating device 15, reaches the positioning area 6 and therefore the body part 7.

To achieve joint pivoting capability of the biopsy device 13 and the light generating device 15, the retaining device 12 is pivotably mounted on the guide carriage 4. The associated pivot axis is similarly a horizontal axis perpendicular to the drawing plane with regard to FIG. 1, running through the positioning area 6. To allow the biopsy to be performed and a reflection-free transmission of the light from the light generating device 15 to the positioning area 6, the retaining device 12 must be pivoted such that the biopsy needle 14 is laterally retractable between the clamping table 8 and the clamping plate 9 in the positioning area 6 in its longitudinal direction 40. To this end the longitudinal direction 40 of the biopsy needle 14 must therefore be pivoted from the vertical position shown in FIG. 1 to a substantially horizontal position. Thus, in the context of the method it is provided that light generated via the light generating device 15 reaches the positioning area 6 and therefore the body part 7 positioned there.

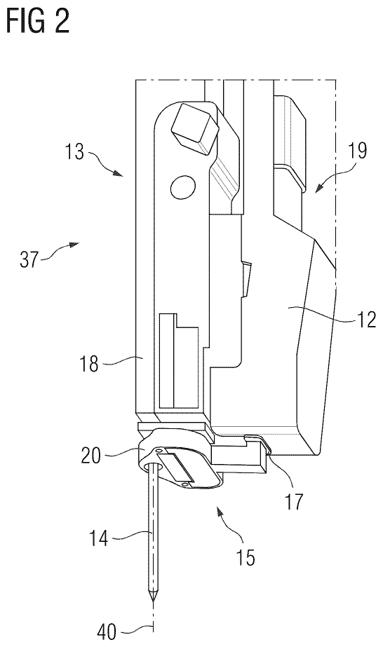

FIG. 2 shows a perspective view of the retaining device 12 together with the biopsy device 13 and the light generating device 15. The assembly or components shown in FIG. 2 form a biopsy unit 37 according to an exemplary embodiment. This is attachable or, with respect to the state shown in FIG. 2, attached to the remaining portion of the medical imaging device 1. Specifically, the biopsy unit 37 is attached to the guide carriage 4 in a detachable manner. The biopsy device 13 and the light generating device 15 are detachably mounted on the retaining device 12. To this end the retaining device 12 has a biopsy device-retaining section for the biopsy device and a light generating device-retaining section for the light generating device 15, the respective device 13, 15 being positioned on and attached in a detachable manner to the respective retaining section. This allows exchangeability of the respective devices 13, 15, in particular for sterility purposes. The biopsy device 13 and the light generating device 15 are in each case attached in a detachable manner to the retaining device 12. In relation to the biopsy device 13, although not shown in more detail in the figures, to this end an attachment means creating a clip or latching connection, interacting by latching with a correspondingly formed latching section of a housing 18 of the biopsy device 13, is conceivable. For attaching the light generating device 15 to the retaining device 12 an attachment means 17 creating a screw connection is for example provided.

A further aspect concerning the biopsy device 13 is the fact that the biopsy needle 14 is movable in its longitudinal direction 40. This allows the biopsy needle 14, once the retaining device 12 in accordance with that explained above has been pivoted into a horizontal position, with regard to FIG. 1, to be introduced or inserted laterally from the left or right into the breast or the body part 7. To this end the biopsy needle 14 is for example extendable from the housing 18. In the exemplary embodiment shown here, however, it is provided that for this purpose the entire retaining device 12 together with the biopsy device 13 and the light generating device 15 is linearly displaceable manually by the doctor. To this end this assembly is movably mounted via a linear guide 19.

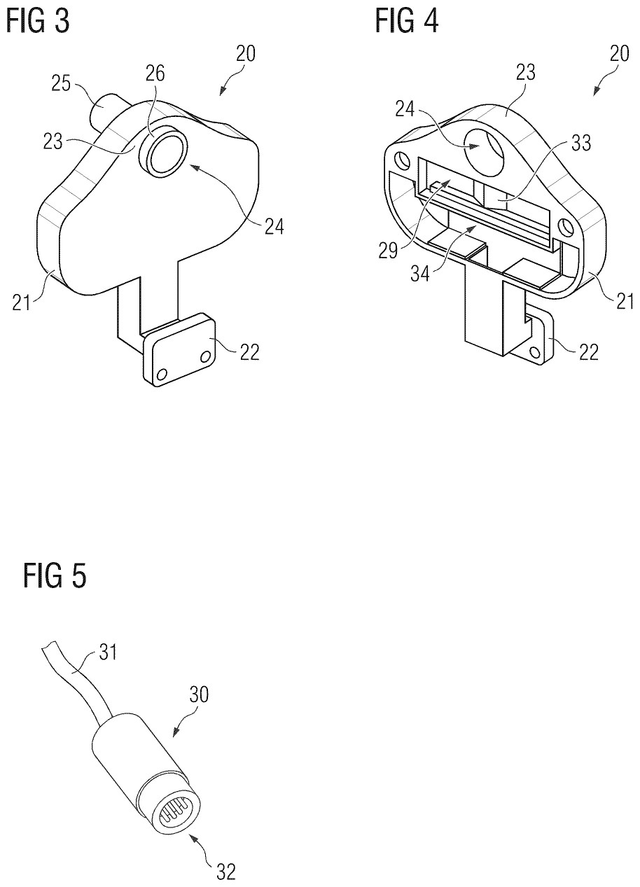

Details in relation to the light generating device 15 are explained below. Thus, in the context of the present exemplary embodiment of the method it is provided that via the light generating device 15 a light reaching the positioning area 6 is generated, details of this being explained below. The light generating device 15 comprises a retaining element 20, FIGS. 3 and 4 showing respective perspective views of the retaining element 20, namely FIG. 3 from the above and FIG. 4 from below. Thus, the retaining element 20 comprises a plate-like body 21 and an attachment section 22 molded onto this, via which the retaining element 20 is attached to the retaining device 12. Purely by way of example, the attachment section 22 in this case has drill holes for receiving the screws provided for in the context of the attachment means 17, so that the attachment section 22 may also be referred to as a screw-on flange. Although in the exemplary embodiment shown the position of the attachment section 22 is elbowed or offset in relation to the plate-like body 21, these sections may also be at the same height or within the same plane. The retaining element 20 is a single piece made of plastic, but may equally also comprise a number of pieces.

On the end facing away from the attachment section 22 the body 21 has a protruding or projecting section 23, in the area of which a guide element-receiving section 24 is provided, which in this case is a drill hole passing through the body 21. In the guide element-receiving section 24 a guide element 25 designed as a guide bushing is inserted, the biopsy needle 14 being positionally stabilized via the guide element 25 in relation to a lateral movement with regard to its longitudinal direction 40. FIG. 3 shows the retaining element 20 with the guide element 25 and FIG. 4 without the guide element 25. Via a laterally protruding end collar 26, which rests on the top of the body 21, in particular with the additional formation of a lateral clamping, the guide element 25 is held in position in the guide element-receiving section 24. The dimensions of the protruding or projecting section 23 are as small as possible, with its extension in relation to the direction pointing away from the attachment section 22 being only slightly larger than the diameter of the drill hole forming the guide element-receiving section 24. Accordingly, the protruding or projecting section 23 in the context of the imaging or X-ray radiation causes as little as possible, if any, shadowing.

The light generating device 15 comprises a marking device 27 and a lighting device 28 (see FIG. 1). Via the marking device 27, in accordance with the exemplary embodiment of the method, a marker light is generated via a laser light and is therefore concentrated or focused, via which a site on the body part 7 positioned in the positioning area 6 is marked in a targeted manner. On the other hand, via the lighting device 28, in accordance with the exemplary embodiment of the method an illuminating light is generated for illuminating a large area of the body part 7 positioned in the positioning area 6.

FIG. 4 shows the retaining element 20 without the devices 27, 28. The retaining element 20 comprises a marking device receiving section 29, in which two laser sources 30 of the marking device 27 are incorporated. The marking device receiving section 29 forms a receiving compartment, running transversally in the body 21. The geometric form of the receiving compartment is adapted to the geometric form of the laser sources 30 or corresponds to these, so that the laser sources 30 are arranged free of play in the marking device receiving section 29. The variants of the retaining element 20 shown in FIGS. 3 and 4 differ slightly from one another in relation to the marking device receiving section 29. Whereas the marking device receiving section 29 in FIG. 3 merely forms a recessed receiving compartment, in FIG. 4 this forms an opening in the body 21.

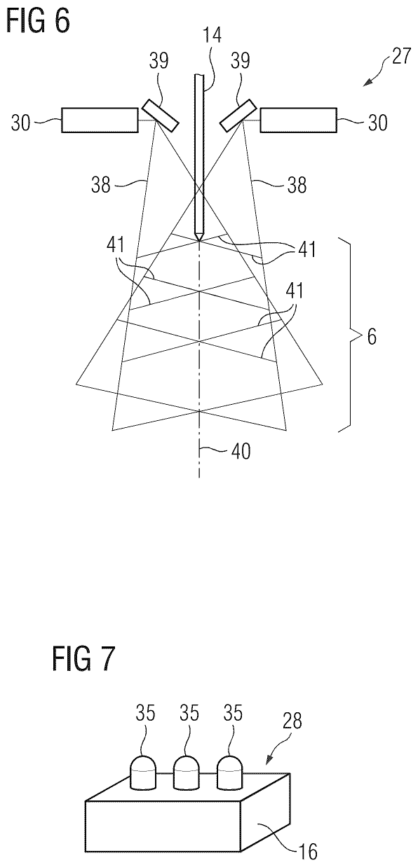

FIG. 5 shows a perspective view of one of the two laser sources 30, which in this case has an elongated and cylindrical form. Also apparent are a power cable 31 and an opening 32, via which the light beam 38 generated via the laser source 30 emerges from this. For a better understanding of the geometric conditions FIG. 6 shows a view of both light beams 38, the propagation planes of which are each at an angle to the drawing plane of FIG. 6. Furthermore, FIG. 6 shows the components of the marking device 27, namely the laser sources 30, and schematically the two mirror surfaces 39 of a mirror 33 at an angle to one another. In the state shown in FIG. 6, these components are arranged in the marking device receiving section 29 of the retaining element 20, which is omitted from this figure for the sake of clarity. Further apparent is the biopsy needle 14, the longitudinal direction 40 of which runs within the drawing plane of FIG. 6.

The two laser sources 30 are structurally identical. Via each of the laser sources 30 a light beam 38 may be generated as a laser light, which propagates in a fan-like way within a respective propagation plane. This means that the geometric form of the light beam 38 corresponds to an equilateral triangle with the laser source 30 at the apex. The two laser sources 30 are arranged in the marking device receiving section 29 in such a way that their openings 32 are facing one another. Between the laser sources 30, the mirror 33 is arranged via which the initially horizontal direction of propagation of the light beam 38 is deflected downwards towards the positioning area 6 and vice versa.

The mirror 33, viewed from the front of the retaining element 20 and therefore perpendicular to the drawing plane of FIG. 1, is V-shaped. Each of the two sides of this V-shape creates one of the mirror surfaces 39, on which the respective light beam 38 of one of the sources 30 is reflected. It is also conceivable for the two mirror surfaces 39 of the mirror 33 to be spaced apart. With regard to FIG. 4, for instance, in this case the mirror surfaces 39 do not touch, but are spaced apart from one another within the marking device receiving section 29, in particular in the longitudinal direction thereof, as indicated accordingly in FIG. 6. The laser sources 30 are rotated relative to one another in the marking device receiving section 29 about their longitudinal axis, so that the propagation planes of the two light beams 38 generated via the laser sources 30, are angled, for example perpendicular, to one another and intersect in an intersection line. The biopsy needle 14, the mirror 33 and the laser sources 30 area arranged in relation to one another in such a way that the longitudinal direction 40 of the biopsy needle 14 lies in each of the two propagation planes, so that the line of intersection of the two propagation planes or light beams 38 coincides after passing the mirror 33 with the longitudinal direction 40 of the biopsy needle 14.

The marker light or the two light beams 38 generate a cross-shaped pattern on the body part 7, the intersection point of the two marking lines 41 of this cross, respectively formed by one of the two light beams 38, marking the site at which the biopsy needle 14 pierces the body part 7 during displacement in its longitudinal direction 40. FIG. 6 shows three pairs of marking lines 41 respectively forming the cross, which in each case are at different heights in the positioning area 6.

In relation to the lighting device 28 it is provided that this is incorporated in a lighting device receiving section 34. The lighting device receiving section 34 is similarly a receiving compartment recessed in the body 21 (see FIG. 4). Via the lighting device 28 a polychromatic, namely white, illuminating light may be generated for illuminating the body part 7 positioned in the positioning area 6. Compared to the marker light a light beam forming the illuminating light fans out all around its direction of propagation and is therefore unfocused. Consequently, the illuminating light is not used to mark a site on the body part 7, but instead for better illumination of the positioning area 6.

FIG. 7 is a highly schematic view of the lighting device 28, which in this case comprises a longitudinal holder 16 with multiple light sources 35, for example provided as light-emitting diodes. The light sources 35 may be light-emitting diodes, for instance SMD light-emitting diodes. Electrical contact points, not shown in more detail, may be provided on the holder 16 to supply the light sources 35. The lighting device 28 may alternatively have just, in particular exclusively, the light sources 35. Thus, the light sources 35 may be inserted directly in the lighting device receiving section 34, in particular soldered in.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A medical imaging device for examination of a body part of a patient, the medical imaging device comprising:
an imaging unit configured to perform an X-ray-based imaging concerning the body part positioned in a positioning area of the medical imaging device; and
a biopsy device comprising a biopsy needle configured to obtain a tissue sample from the body part positioned in the positioning area and a light generating device configured to generate light to reach the positioning area.

2. The medical imaging device of claim 1, wherein the light generating device comprises a marking device configured to generate a marker light for targeted marking of a site on the body part positioned in the positioning area.

3. The medical imaging device of claim 2, wherein the biopsy needle is movable in a longitudinal direction of the biopsy needle to bring the biopsy needle into a state of insertion in the body part positioned in the positioning area, wherein the site marked via the marker light is the site at which the longitudinal direction of the biopsy needle impinges on the body part positioned in the positioning area.

4. The medical imaging device of claim 3, wherein the marker light is formed by a first light beam, propagating in a fan within a first propagation plane, and by a second light beam, propagating in a fan within a second propagation plane, the first propagation plane and the second propagation plane being at an angle to one another, wherein the longitudinal direction of the biopsy needle lies within each of the first propagation plane and the second propagation plane.

5. The medical imaging device of claim 2, wherein the light generating device includes at least one mirror, wherein the at least one mirror is configured to deflect a direction of propagation of the marker light towards the positioning area.

6. The medical imaging device of claim 1, wherein the light generating device comprises a lighting device, the lighting device configured to generate an illuminating light to illuminate the body part positioned in the positioning area.

7. The medical imaging device of claim 2, wherein the light generating device includes a retaining element with at least one marking device receiving section to receive the marking device.

8. The medical imaging device of claim 7, wherein the marking device receiving section is a receiving compartment recessed into or penetrating a body of the retaining element.

9. The medical imaging device of claim 7, wherein a guide element is incorporated in a guide element-receiving section of the retaining element, and the biopsy needle is positionally stabilized via the guide element.

10. The medical imaging device of claim 1, further comprising:
a retaining device, the biopsy device and the light generating device being on the retaining device.

11. The medical imaging device of claim 10, wherein the retaining device is repositionable to jointly reposition the biopsy device and the light generating device in relation to the positioning area.

12. The medical imaging device of claim 1, further comprising:
a positioning device arranged at or in the positioning area, the positioning device configured to position the body part in the positioning area for performing the examination.

13. The medical imaging device of claim 1, wherein the imaging unit includes an X-ray source configured to generate incident X-ray radiation in the positioning area and an X-ray detector configured to detect the X-ray radiation passing through the positioning area, wherein the X-ray source is positionable in relation to the positioning area.

14. A biopsy unit for a medical imaging device for examination of a body part of a patient, wherein the medical imaging device comprises an imaging unit configured to perform X-ray-based imaging concerning the body part positioned in a positioning area of the medical imaging device, the biopsy unit comprising:
a biopsy device including,
a biopsy needle configured to obtain a tissue sample from the body part positioned in the positioning area, and
a light generating device configured to generate light for the positioning area with respect to a state in which the biopsy unit is mounted on a remaining portion of the medical imaging device.

15. A method for illuminating a body part of a patient using the medical imaging device of claim 1, the method comprising:
positioning the body part in the positioning area; and
generating, by the light generating device, the light reaching the positioning area.

16. The medical imaging device of claim 1, wherein the medical imaging device is a mammography device.

17. The medical imaging device of claim 6, wherein the light generating device includes at least one lighting device receiving section to receive the lighting device.

18. The medical imaging device of claim 17, wherein the lighting device receiving section is a receiving compartment recessed into or penetrating a body of a retaining element.

19. The medical imaging device of claim 13, wherein the X-ray source is pivotable in relation to the positioning area.
